(19) United States
(12) Patent Application Publication
(10) Pub. No.: US 2021/0338666 A1
(43) Pub. Date: Nov. 4, 2021

(54) BRIMONIDINE COMBINATIONS AND USES THEREOF

(71) Applicant: Eye Therapies, LLC, Dana Point, CA (US)

(72) Inventor: Gerald Horn, Deerfield, IL (US)

(21) Appl. No.: 17/244,094

(22) Filed: Apr. 29, 2021

Related U.S. Application Data
(60) Provisional application No. 63/017,778, filed on Apr. 30, 2020.

(51) Int. Cl. A61K 31/4985; A61K 9/00; A61K 47/02; A61P 27/02; A61K 47/38; A61K 47/26; A61K 47/40; A61K 47/10; A61K 47/18; A61K 47/12

(52) U.S. Cl. CPC A61K 31/4985; A61K 9/0048; A61K 47/02; A61P 27/02; A61K 47/12; A61K 47/26; A61K 47/40; A61K 47/10; A61K 47/183; A61K 47/38

(57) ABSTRACT

The present invention is directed to brimonidine compositions for reducing eye redness, increasing eye whiteness and/or reducing rebound hyperemia. The present invention is further directed to methods of reducing eye redness, increasing eye whiteness and/or reducing rebound hyperemia comprising applying brimonidine compositions to an eye of a subject in need thereof.

### FIELD OF THE INVENTION

The present invention is directed to brimonidine compositions for reducing eye redness, increasing eye whiteness and/or reducing rebound hyperemia. The present invention is further directed to methods of reducing eye redness, increasing eye whiteness and/or reducing rebound hyperemia comprising applying brimonidine compositions to an eye of a subject in need thereof.

### BACKGROUND OF THE INVENTION

Dilation of small blood vessels, particularly arterioles, capillaries, and venules, causes many clinically undesirable events including surface hemorrhage and hyperemia following Lasik surgery, eye redness (conjunctival hyperemia), and nasal congestion (turbinate mucosal swelling secondary to vasodilation).

Adrenergic receptors mediate physiological responses to the catecholamines, norephinephrine and epinephrine, and are members of the superfamily of G protein-coupled receptors having seven transmembrane domains. These receptors, which are divided pharmacologically into α-1, α-2 and β-adrenergic receptor types, are involved in diverse physiological functions including functions of the cardiovascular and central nervous systems. The α-adrenergic receptors mediate excitatory and inhibitory functions: α-1 adrenergic receptors are typically excitatory post-synaptic receptors which generally mediate responses in an effector organ, while α-2 adrenergic receptors are located postsynaptically as well as presynaptically, where they inhibit release of neurotransmitters. Agonists of α-2 adrenergic receptors currently are used clinically in the treatment of hypertension, glaucoma, spasticity, and attention-deficit disorder, in the suppression of opiate withdrawal, as adjuncts to general anesthesia and in the treatment of cancer pain. Vascular constriction is known to be mediated by α-adrenergic receptors.

α-2 adrenergic receptors are presently classified into three subtypes based on their pharmacological and molecular characterization: α-2A/D (α-2A in human and α-2D in rat); α-2B; and α-2C (Bylund et al., Pharmacol. Rev. 46:121-136 (1994); and Hein and Kobilka, Neuropharmacol. 34:357-366 (1995)). The α-2A, α-2B, and α-2C subtypes appear to regulate arterial and/or venular contraction in some vascular beds, and the α-2A and α-2C subtypes mediate feedback inhibition of norepinephrine release from sympathetic nerve endings. The α-2A subtype also mediates many of the central effects of α-2 adrenergic agonists (Calzada and ArtiZano, Pharmacol. Res. 44: 195-208 (2001); Hein et al., Ann. NY Acad. Science 881:265-271 (1999); and Ruffolo (Ed.), α-Adrenoreceptors: Molecular Biology, Biochemistry and Pharmacology S. Karger Publisher's Inc. Farmington, Conn. (1991)). The α-2A subtype also mediates potent constriction of the porcine, but not human, ciliary artery.

Many compounds having selective α-2 agonist activity are known and include brimonidine (which has been used for lowering intraocular pressure in patients with open-angle glaucoma or ocular hypertension), guanfacine (which has been used to control high blood pressure), dexmetidomidine (which has been used as a sedative, analgesic, sympatholytic and anxiolytic), and methyl dopa (which has been used as a centrally-acting adrenergic antihypertensive).

The clinically available compounds belong to the general category of a adrenergic receptor agonists. It is a known property of all a adrenergic receptor agonists, including brimonidine, to cause vasoconstriction. However, known formulations of brimonidine and other known α-2 adrenergic receptor agonists are associated with a high incidence of rebound hyperemia, or other side effects, in clinical use. For example, after as few as three doses of applying known formulations of a adrenergic receptor agonists, patients may develop secondary rebound hyperemia or secondary vasodilation. Brimonidine (5-bromo-6-(2-imidazolidinylideneamino) quinoxaline L-tartrate), a known selective alpha 2 agonist is associated with significant rebound hyperemia (primary or delayed onset vasodilation) in its current concentration range for treating glaucoma of about 0.1% to 0.2%.

Commercially available general alpha agonists for topical mucosal decongestant use (ophthalmic and nasal applications) include tetrahydrozoline, naphazoline, oxymetazoline, xylometazoline, methoxamine and phenylephrine. These agonists have high alpha 1 receptor agonist activity and are known to cause rebound hyperemia and medicamentosa. Accordingly, their clinical use is usually restricted to several hours or a few days, at most. Many individuals with mucosal congestion or hyperemia from chronic conditions such as dry eye, contact lens wear, allergic conjunctivitis, allergic rhinitis, nonallergic rhinitis, acute or chronic sinusitis, nasal polyposis, rhinitis secondary to pregnancy, or rhinitis due to nasal septal deviation or obstruction and asthma, particularly, allergic asthma require longer term agonist use.

Recently, Lumify®, 0.025% brimonidine tartrate eye drop was approved by the Food and Drug Administration for reducing redness (Lumify is a registered trademark of and available from Bausch and Lomb). Lumify® is described in and/or covered by U.S. Pat. Nos. 8,338,421, 8,987,270, 8,283,350, 8,580,787, 8,293,742, 8,765,758 and 9,259,425. However, Lumify® may be slightly uncomfortable for some users including those experiencing dry eyes. Further, contact lens users may prefer a preservative free formulation.

Thus, there is a need for more comfortable or preservative free formulations that continue to provide safe and long term vasoconstriction.

### SUMMARY OF THE INVENTION

In one embodiment, the present invention is directed to ophthalmological compositions for reducing eye redness, increasing eye whiteness and/or reducing rebound hyperemia comprising, consisting essentially of or consisting of brimonidine or a salt thereof.

In a preferred embodiment, compositions of the present invention comprise, consist essentially of or consist of brimonidine or a salt thereof and one or more excipients selected from the group consisting of a surfactant, a viscosity enhancer, a tonicity adjustor, a preservative and a buffer.

In one embodiment, the present invention is directed to ophthalmological compositions for reducing eye redness, increasing eye whiteness and/or reducing rebound hyperemia comprising, consisting essentially of or consisting of brimonidine or a salt thereof and one or more excipients selected from the group consisting of a surfactant, a viscosity enhancer, a tonicity adjustor, a preservative, a buffer and a sugar.

In one embodiment, the present invention is directed to ophthalmological compositions for reducing eye redness, increasing eye whiteness and/or reducing rebound hyperemia comprising, consisting essentially of or consisting of brimonidine or a salt thereof and one or more excipients selected from the group consisting of a surfactant, a polyethylene glycol, a viscosity enhancer, a tonicity adjustor, a preservative and a buffer.

In one embodiment, the present invention is directed to ophthalmological compositions for reducing eye redness, increasing eye whiteness and/or reducing rebound hyperemia comprising, consisting essentially of or consisting of brimonidine or a salt thereof and one or more excipients selected from the group consisting of a surfactant, a polyethylene glycol, a viscosity enhancer, a tonicity adjustor, a preservative, a buffer and a sugar.

In another embodiment, the present invention is directed to ophthalmological compositions for contact lenses for reducing eye redness, increasing eye whiteness and/or reducing rebound hyperemia comprising, consisting essentially of or consisting of brimonidine or a salt thereof and one or more excipients selected from the group consisting of a tonicity adjustor and a buffer, wherein optionally, the composition does not contain a preservative.

In another embodiment, the present invention is directed to a method selected from selected from the group consisting of reducing eye redness, increasing eye whiteness and reducing rebound hyperemia comprising applying a composition of the present invention to an eye of a subject in need thereof.

In another embodiment, the present invention is directed to a method selected from the group consisting of reducing eye redness, increasing eye whiteness and reducing rebound hyperemia comprising applying a composition of the present invention to a contact lens and inserting the contact lens into an eye of a subject in need thereof or applying a composition of the present invention to an eye of a subject in need thereof wherein the eye comprises a contact lens.

### DETAILED DESCRIPTION OF THE INVENTION

The present invention is directed to extremely low dose brimonidine compositions. These compositions may be used to reduce eye redness, increase eye whiteness and/or to reduce rebound hyperemia.

In one embodiment, the present invention is directed to ophthalmological compositions for reducing eye redness, increasing eye whiteness and/or reducing rebound hyperemia comprising, consisting essentially of or consisting of brimonidine or a salt thereof.

In a preferred embodiment, compositions of the present invention comprise, consist essentially of or consist of brimonidine or a salt thereof and one or more excipients selected from the group consisting of a surfactant, a viscosity enhancer, a tonicity adjustor, a preservative and a buffer.

In one embodiment, the present invention is directed to ophthalmological compositions for reducing eye redness, increasing eye whiteness and/or reducing rebound hyperemia comprising, consisting essentially of or consisting of brimonidine or a salt thereof and one or more excipients selected from the group consisting of a surfactant, a viscosity enhancer, a tonicity adjustor, a preservative, a buffer and a sugar.

In one embodiment, the present invention is directed to ophthalmological compositions for reducing eye redness, increasing eye whiteness and/or reducing rebound hyperemia comprising, consisting essentially of or consisting of brimonidine or a salt thereof and one or more excipients selected from the group consisting of a surfactant, a polyethylene glycol, a viscosity enhancer, a tonicity adjustor, a preservative and a buffer.

In one embodiment, the present invention is directed to ophthalmological compositions for reducing eye redness, increasing eye whiteness and/or reducing rebound hyperemia comprising, consisting essentially of or consisting of brimonidine or a salt thereof and one or more excipients selected from the group consisting of a surfactant, a polyethylene glycol, a viscosity enhancer, a tonicity adjustor, a preservative, a buffer and a sugar.

In another embodiment, the present invention is directed to ophthalmological compositions for contact lenses for reducing eye redness, increasing eye whiteness and/or reducing rebound hyperemia comprising, consisting essentially of or consisting of brimonidine or a salt thereof and one or more excipients selected from the group consisting of a tonicity adjustor and a buffer, wherein optionally, the composition does not contain a preservative.

In a preferred embodiment, compositions of the present invention do not contain aceclidine.

In a preferred embodiment, brimonidine or a salt thereof is present in compositions of the present at a concentration of less than about 0.001% w/v, more preferably less than about 0.0009% w/v, even more preferably less than 0.0008% w/v and even more preferably about 0.00085% w/v or less, even more preferably about 0.00075% w/v or less and even more preferably at about 0.00075% w/v.

In another preferred embodiment, brimonidine or a salt thereof is present in compositions of the present at a concentration of from about 0.00001% to about 0.0009% w/v, more preferably from about 0.00001% to about 0.00085% w/v, even more preferably from about 0.00001% to about 0.0008% w/v, and even more preferably from about 0.00001% to about 0.00075% w/v.

Surfactants suitable for use in the present invention include, but are not limited to, polysorbates, polyoxyls, poloxamers, cyclodextrins and combinations thereof. In a preferred embodiment, the surfactant is a polysorbate, a cyclodextrin or a combination thereof. In a more preferred embodiment, the surfactant is polysorbate 80, hydroxypropyl gamma cyclodextrin or a combination thereof.

Surfactants may be present in compositions of the present invention at a concentration from about 0.1% to about 10% w/v, preferably from about 1% to about 10% w/v, even more preferably from about 1% to about 4% w/v, from about 2% to about 4% w/v or from about 0.5% to about 1.5% w/v. In a most preferred embodiment, the surfactant is present in compositions of the present invention at a concentration of about 1%, about 3% or about 4% w/v.

Viscosity enhancers suitable for use in the present invention include, but are not limited to, cellulose derivatives, carbomers, gums, and hyaluronic acids, dextrans, polyvinyl alcohol, polyacrylic acids, povidone, polyethylene glycols, propylene glycol, chitosans and combinations thereof. In a preferred embodiment, the viscosity enhancer is a cellulose derivative. In a more preferred embodiment, the viscosity enhancer is carboxymethyl cellulose.

Viscosity enhancers may be present in compositions of the present invention at a concentration from about 0.1% to about 10% w/v, preferably from about 0.5% to about 5% w/v, even more preferably from about 1% to about 2% w/v and yet even more preferably at about 1.45% w/v.

Tonicity adjustors suitable for use in the present invention include, but are not limited to, a salt, a polyol, glycerin or a combination thereof. In a preferred embodiment, the tonicity adjustor is a salt, more preferably sodium chloride.

Tonicity adjustors may be present in compositions of the present invention at a concentration from about 0.001% to about 1% w/v, preferably from about 0.01% to about 0.1% w/v, even more preferably from about 0.05% to about 0.1% w/v, yet even more preferably from about 0.08% to about 0.1% w/v and most preferably at about 0.09% w/v.

Polyethylene glycols suitable for use in the present invention include, but are not limited to, polyethylene glycols having a molecular weight from about 400 to about 20,000 Daltons including polyethylene glycol 400 and polyethylene glycol 20000.

Polyethylene glycol may be present in compositions of the present invention at a concentration from about 0.1% to about 5% w/v, preferably from about 0.1% to about 1% w/v, even more preferably from about 0.1% to about 0.5% w/v and yet even more preferably from about 0.2% to about 0.3% w/v. In a most preferred embodiment, the polyethylene glycol is present in compositions of the present invention at a concentration of about 0.25% w/v.

Sugars suitable for use in the present invention include, but are not limited to, glucose, sucrose, trehalose, lactose, maltose, fructose, ribose and dextran. In a most preferred embodiment, the sugar is D-ribose.

Sugars may be present in compositions of the present invention at a concentration from about 0.001% to about 1% w/v, preferably from about 0.01% to about 0.1% w/v, even more preferably from about 0.05% to about 0.1% w/v, yet even more preferably from about 0.08% to about 0.1% w/v and most preferably at about 0.09% w/v.

Preservatives suitable for use in the present invention include, but are not limited to, benzalkonium chloride, sorbic acid, potassium sorbate, oxychloro complex, citric acid, chlorobutanol, thimerosal, phenylmercuric acetate, disodium ethylenediaminetetraacetic acid (“EDTA”), phenylmercuric nitrate, perborate, benzyl alcohol and combinations thereof. In a preferred embodiment the preservative is potassium sorbate, EDTA or a combination thereof.

Preservatives may be present in compositions of the present invention at a concentration from about 0.001% to about 1% w/v, preferably from about 0.001% to about 0.5% w/v, even more preferably from about 0.01% to about 0.13% w/v, yet even more preferably from about 0.01% to about 0.11% w/v, and yet even more preferably from about 0.005% to about 0.015% w/v, from about 0.0075% to about 0.0125% w/v, from about 0.05% to about 0.15% w/v, or from about 0.075% to about 0.125% w/v. In a most preferred embodiment, the preservative is at a concentration of about 0.01%, about 0.1% or about 0.11% w/v.

Various buffers and means for adjusting pH can be used to prepare ophthalmological compositions of the invention. Such buffers include, but are not limited to, acetate buffers, citrate buffers, phosphate buffers and borate buffers. In a preferred embodiment, the buffer is acetate buffer or phosphate buffer. It is understood that acids or bases can be used to adjust the pH of the composition as needed.

Buffers may be present in compositions of the present invention at concentration from 1 to 10 millimolar (“mM”), preferably from about 2 to about 5 mM, more preferably from about 2.5 to about 3.0 mM and most preferably at about 2.5 or 3.0 mM. In a preferred embodiment the compositions of the pH is from about 4.0 to about 8.0, more preferably from about 5.0 to about 7.0 and even more preferably from about 5.5 to about 7.0. In a most preferred embodiment, the composition has a pH of about 5.5 or 7.0.

In a preferred embodiment, the present invention is directed to an ophthalmic composition comprising:

about 0.00075% w/v brimonidine or a salt thereof;

about 3.0% w/v polysorbate 80;

about 1.45% w/v carboxymethyl cellulose;

about 0.09% w/v sodium chloride;

about 2.5 mM acetate buffer;

about 0.1% w/v EDTA; and

about 0.01% w/v potassium sorbate,

wherein the composition has a pH of about 5.5.

In another preferred embodiment, the present invention is directed to an ophthalmic composition comprising:

about 0.00075% w/v brimonidine or a salt thereof;

about 3.0% w/v polysorbate 80;

about 1.45% w/v carboxymethyl cellulose;

about 0.09% w/v sodium chloride;

about 2.5 mM acetate buffer;

about 0.1% w/v EDTA;

about 0.01% w/v potassium sorbate; and

about 0.09% w/v D-ribose,

wherein the composition has a pH of about 5.5.

In another preferred embodiment, the present invention is directed to an ophthalmic composition comprising:

about 0.00075% w/v brimonidine or a salt thereof;

about 0.09% w/v sodium chloride; and

about 3.0 mM phosphate buffer,

wherein the composition does not contain a preservative and has a pH of about 7.0.

In another preferred embodiment, the present invention is directed to an ophthalmic composition comprising:

about 0.00075% w/v brimonidine or a salt thereof;

about 3.0% w/v polysorbate 80;

about 1.45% w/v carboxymethyl cellulose;

about 0.25% w/v polyethylene glycol 400;

about 1.0% w/v hydroxypropyl gamma cyclodextrin;

about 0.09% w/v sodium chloride;

about 2.5 mM acetate buffer;

about 0.1% w/v EDTA; and

about 0.01% w/v potassium sorbate,

wherein the composition has a pH of about 5.5.

In another preferred embodiment, the present invention is directed to an ophthalmic composition comprising:

about 0.00075% w/v brimonidine or a salt thereof;

about 3.0% w/v polysorbate 80;

about 1.45% w/v carboxymethyl cellulose;

about 0.25% w/v polyethylene glycol 400;

about 1.0% w/v hydroxypropyl gamma cyclodextrin;

about 0.09% w/v sodium chloride;

about 2.5 mM acetate buffer;

about 0.1% w/v EDTA;

about 0.01% w/v potassium sorbate; and

about 0.09% w/v D-ribose,

wherein the composition has a pH of about 5.5.

The compositions of the present invention may in the form of a liquid, gel, ointment or cream. Compositions of the present invention may be applied to the eye of a subject in need thereof by any available means including, but not limited to, a drop or a spray. The subject may be wearing a contact lens during instillation of the compositions of the present invention. Compositions of the present invention may also be placed on a contact lens that is then inserted in the eye of a subject in need thereof.

In another embodiment, the present invention is directed to a method selected from selected from the group consisting of reducing eye redness, increasing eye whiteness and reducing rebound hyperemia comprising applying a composition of the present invention to an eye of a subject in need thereof.

In another embodiment, the present invention is directed to a method selected from the group consisting of reducing eye redness, increasing eye whiteness and reducing rebound hyperemia comprising applying a composition of the present invention to a contact lens and inserting the contact lens into an eye of a subject in need thereof or applying a composition of the present invention to an eye of a subject in need thereof wherein the eye comprises a contact lens.

As used herein, the terms “reduce” or “reducing” refer to decreasing the surface area covered or lessening the intensity.

As used herein, the terms “increase” or “increasing” refer to increasing the surface area covered or increasing the intensity. When referring to “whiteness” the terms “increase” and “increasing” refer to increasing the surface area covered or intensity of whiteness beyond a baseline whiteness. Baseline whiteness is measured when the subject is not suffering from hyperemia.

As used herein, the terms “reduce” or “reducing” refer to decreasing the duration or intensity.

As used herein “salts” refers to those salts which retain the biological effectiveness and properties of the parent compounds and which are not biologically or otherwise harmful at the dosage administered. Salts of the compounds of the present inventions may be prepared from inorganic or organic acids or bases.

The compounds of the present invention can be used in the form of pharmaceutically acceptable salts derived from inorganic or organic acids or bases. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq.

The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, hyaluronic acid, malic acid, sulphuric acid and phosphoric acid and such organic acids as oxalic acid, malic acid, maleic acid, methanosulfonic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethyl ammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

As used herein, all numerical values relating to amounts, weights, and the like, that are defined as “about” each particular value is plus or minus 10%. For example, the phrase “about 5% w/v” is to be understood as “4.5% to 5.5% w/v.” Therefore, amounts within 10% of the claimed value are encompassed by the scope of the claims.

As used herein “% w/v” refers to the percent weight of the total composition.

As used herein the term “subject” refers but is not limited to a person or other animal.

### EXAMPLES

### Example 1-Prophetic Use of a Composition of the Present Invention

### Method

A subject that previously experienced slight discomfort after instilling Lumify® in the subject's eyes had an irritant instilled in each eye causing redness in each eye. The subject was then topically administered Lumify® containing benzalkonium chloride, boric acid, calcium chloride dihydrate, glycerin, potassium chloride, sodium borate decahydrate, sodium chloride, water at various concentrations of brimonidine tartrate as in Table 1, below, in the left eye and Composition #1 containing 3.0% w/v polysorbate 80, 1.45% w/v carboxymethyl cellulose, 0.09% w/v sodium chloride, 2.5 mM acetate buffer, 0.1% w/v EDTA, 0.01% w/v potassium sorbate, water and brimonidine tartrate at concentrations of Table 1, below, in the right eye. A one-week washout period occurred between each set of instillations.

TABLE 1

| | Left Eye | Right Eye |
|---|---|---|
| | (% Brimonidine in | (% Brimonidine in |
| Instillation | Lumify ®) | Composition #1) |
| #1 | 0.01% | 0.00050% |
| #2 | 0.01% | 0.00075% |
| #3 | 0.01% | 0.00085% |
| #4 | 0.005% | 0.00050% |
| #5 | 0.005% | 0.00075% |
| #6 | 0.005% | 0.00085% |
| #7 | 0.0025% | 0.00050% |
| #8 | 0.0025% | 0.00075% |
| #9 | 0.0025% | 0.00085% |

### Results

The subject had similar redness reduction in the left and right eye. Further, the subject also had similar increase in whiteness of the sclera in the left and right eye going from a slightly yellow color to a bright white. However, the subject reported experiencing slight discomfort in the left eye upon instillation and at 1- and 2-hours following instillation. Unexpectedly, the subject experienced no discomfort in the right eye upon instillation or at 1, 2- or 4-hours following instillation. Thus, compositions of the present invention provide increased comfort in subjects that experience discomfort upon instillation of Lumify®.

### Example 2-Prophetic Use of a Composition of the Present Invention in Contact Lens Wearer

### Method

A subject that wears contact lenses was topically administered a preservative-free composition containing 0.00075% w/v brimonidine tartrate, 0.09% w/v sodium chloride; and 3.0 mM phosphate buffer. The pH of the composition was 7.0.

### Results

The subject had comparable redness reduction in the left and right eye. Further, the subject also had comparable increase in whiteness of the sclera in the left and right eye going from a slightly yellow color to a bright white. Thus, preservative-free compositions of the present invention are able to achieve redness reduction and increased whiteness in contact lens wearers.

What is claimed is:

1. An ophthalmological composition for reducing eye redness, increasing eye whiteness and/or reducing rebound hyperemia comprising less than about 0.001% w/v brimonidine or a salt thereof, wherein w/v denotes weight by total volume of the composition.

2. The composition of claim 1, wherein the brimonidine or salt thereof is at a concentration of about 0.00085% w/v or less.

3. The composition of claim 2, wherein the brimonidine or salt thereof is at a concentration of about 0.00075% w/v or less.

4. The composition of claim 1, further comprising one or more excipients selected from a surfactant, a viscosity enhancer, a tonicity adjustor, a preservative and a buffer.

5. The composition of claim 4, comprising one or more excipients selected from the group consisting of from about 0.1% to about 10% w/v of a surfactant, from about 0.1% to about 10% w/v of a viscosity enhancer, from about 0.0001% to about 1% w/v of a tonicity adjustor, from about 0.001% to about 1% of a preservative and from about 1 to about 10 millimolar of a buffer.

6. The composition of claim 4, wherein the surfactant is polysorbate 80, the viscosity enhancer is carboxymethyl cellulose, the tonicity adjustor is sodium chloride, the preservative is a combination of disodium ethylenediaminetetraacetic acid and potassium sorbate.

7. The composition of claim 4, wherein the surfactant is a combination of polysorbate 80 and hydroxypropyl gamma cyclodextrin, the tonicity adjustor is sodium chloride, the preservative is a combination of disodium ethylenediaminetetraacetic acid and potassium sorbate.

8. The composition of claim 1, further comprising a sugar.

9. The composition of claim 8, wherein the sugar is D-ribose.

10. The composition of claim 1, further comprising a polyethylene glycol.

11. The composition of claim 10, wherein the polyethylene glycol has a molecular weight of 400 Daltons.

12. The composition of claim 1, further comprising a tonicity adjustor and a buffer.

13. The composition of claim 12, wherein the tonicity adjustor is sodium chloride and the buffer is phosphate buffer, wherein the composition does not contain a preservative.

14. An ophthalmological composition for reducing eye redness, increasing eye whiteness and/or reducing rebound hyperemia comprising from about 0.0001% to about 0.00075% w/v brimonidine or a salt thereof, from about 2% to about 4% w/v of a surfactant, from about 1% to about 2% w/v of a viscosity enhancer, from about 0.05% to about 0.1% w/v of a tonicity adjustor, from about 0.01% to about 0.13% w/v of a preservative, wherein w/v denotes weight by total volume of the composition.

15. The composition of claim 14, further comprising from about 0.05% to about 0.1% w/v of a sugar.

16. The composition of claim 14, further comprising from about 0.1% to about 0.5% w/v of a polyethylene glycol.

17. An ophthalmological compositions for reducing eye redness, increasing eye whiteness and/or reducing rebound hyperemia comprising:
about 0.00075% w/v brimonidine or a salt thereof;
about 3.0% w/v polysorbate 80;
about 1.45% w/v carboxymethyl cellulose;
about 0.09% w/v sodium chloride;
about 2.5 millimolar acetate buffer;
about 0.1% w/v EDTA; and
about 0.01% w/v potassium sorbate,
wherein the composition has a pH of about 5.5 and w/v denotes weight by total volume of the composition.

18. The composition of claim 17, further comprising about 0.09% w/v D-ribose.

19. The composition of claim 18, further comprising about 1.0% w/v hydroxypropyl gamma cyclodextrin and about 0.25% w/v of a polyethylene glycol having a molecular weight of 400 Daltons.

20. The composition of claim 19, further comprising about 0.09% w/v D-ribose.

21. An ophthalmological composition for reducing eye redness, increasing eye whiteness and/or reducing rebound hyperemia comprising:
about 0.00075% w/v brimonidine or a salt thereof;
about 0.09% w/v sodium chloride; and
about 3.0 millimolar phosphate buffer,
wherein the composition has a pH of about 7.0, the composition does not contain a preservative and w/v denotes weight by total volume of the composition.

22. A method selected from the group consisting of reducing eye redness, increasing eye whiteness and reducing rebound hyperemia comprising applying the composition of claim 1 to an eye of a subject in need thereof.

23. A method selected from the group consisting of reducing eye redness, increasing eye whiteness and reducing rebound hyperemia comprising applying the composition of claim 12 to a contact lens and inserting the contact lens into an eye of a subject in need thereof or applying the composition of claim 1 to an eye of a subject in need thereof wherein the eye comprises a contact lens.
